(19) United States
(12) Patent Application Publication
(10) Pub. No.: US 2011/0109107 A1
(43) Pub. Date: May 12, 2011

(54) Suction cup type contact lens removal device and method of removing a lens by means of same

(76) Inventor: Ping Yi Lin, Taipei (TW)

(21) Appl. No.: 12/614,379

(22) Filed: Nov. 6, 2009

(51) Int. Cl. A61F 9/00

(52) U.S. Cl. 294/1.2

(57) ABSTRACT

A method of removing a contact lens by means of a device including a flexible cup comprising a bottom projection and an outward extending rim on an opening, and a suction loop member shaped to assemble with the rim by snapping is provided. The method includes pouring an amount of IV fluid into the cup; adhering the suction loop member to a periphery of the eye by engaging; immersing the contact lens placed on the cornea of the eye in the IV fluid by inverting the cup; and repeatedly pressing and releasing the cup until the contact lens falls onto the projection. Hand contact with the eye is avoided.

### BACKGROUND OF THE INVENTION

1. Field of Invention

The invention relates to contact lens removal devices and more particularly to a suction cup type contact lens removal device and method of removing a lens by means of same without contacting the lens with the fingers.

2. Description of Related Art

Conventionally, a contact lens is classified as either soft lens or rigid lens. For rigid lens, a user may pull with the outer or lateral canthus and then blink to cause the lens to lose adhesion. The other hand is cupped under the eye to catch the lens.

For soft lens as shown in FIG. 1, a user may hold the eyelid open and grasp the lens with the thumb and the index finger. However, this method may cause irritation, risk damage to the eye, and be difficult. Thus, the need for improvement still exists.

### SUMMARY OF THE INVENTION

It is therefore one object of the invention to provide a contact lens removal device.

It is another object of the invention to provide a method of removing a lens by means of a contact lens removal device without contacting the lens with the fingers.

The above and other objects, features and advantages of the invention will become apparent from the following detailed description taken with the accompanying drawings.

### BRIEF DESCRIPTION OF THE DRAWINGS

FIG. 1 is a side elevation depicting a conventional method of removing a soft lens with the hand;

FIG. 2 is an exploded view of a preferred embodiment of contact lens removal device according to the invention;

FIG. 3 is a longitudinal sectional view of the contact lens removal device shown in FIG. 2; and

FIGS. 4, 5, and 6 are side elevation depicting three lens removal steps by means of the contact lens removal device of the invention.

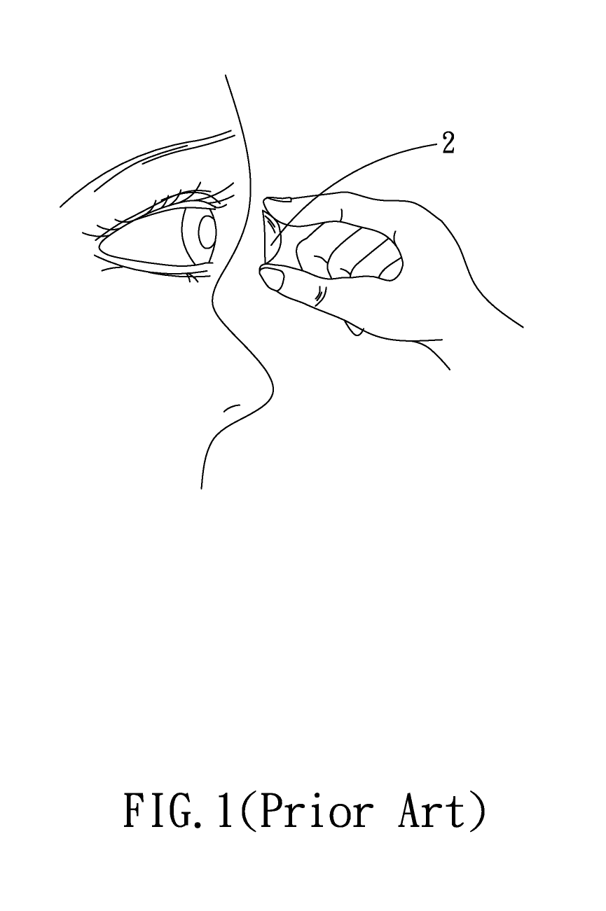

### DETAILED DESCRIPTION OF THE INVENTION

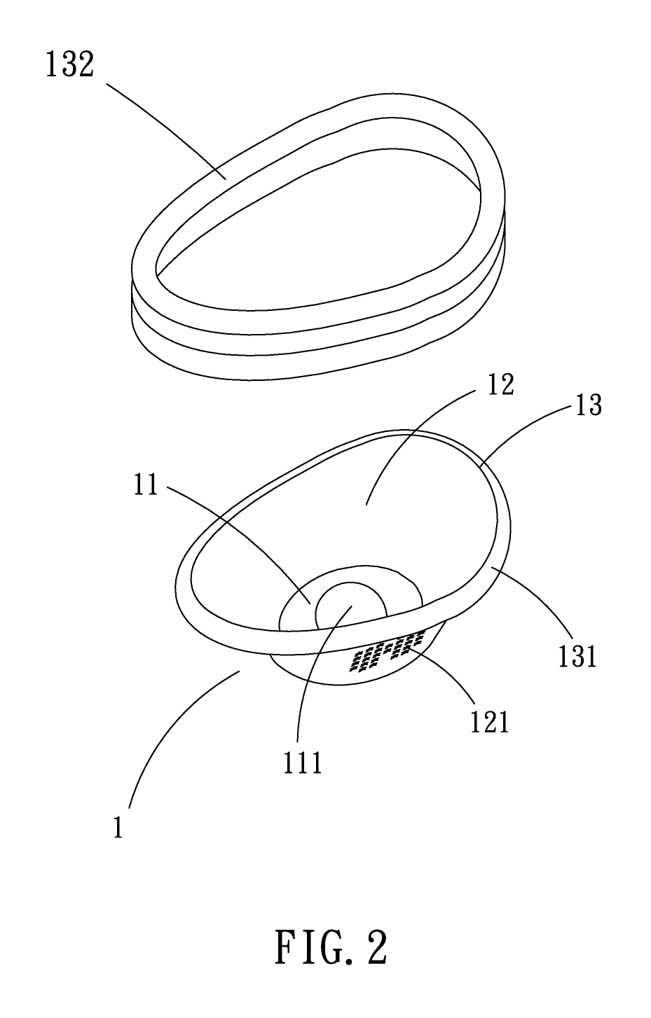

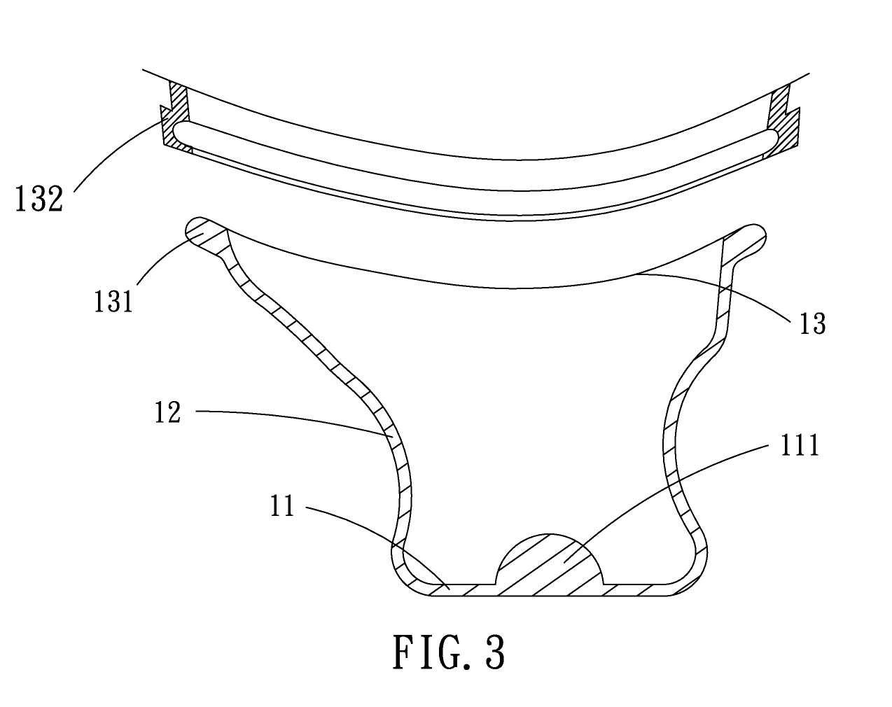

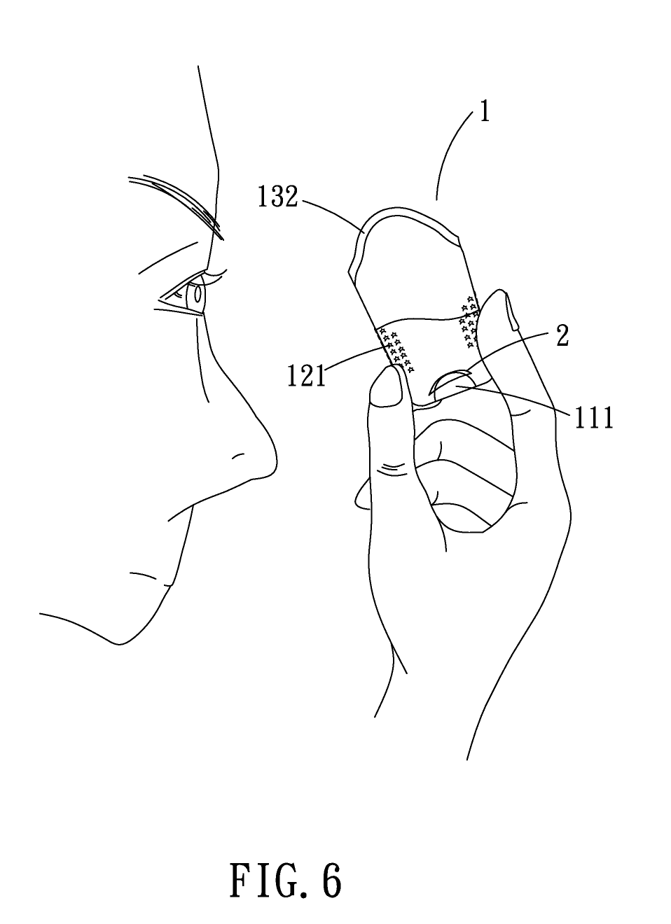

Referring to FIGS. 2 to 6, a contact lens removal device in accordance with a preferred embodiment of the invention comprises the following components as discussed in detail below.

A flexible cup 1 comprises a bottom 11 having a half-spherical projection 111 on a central portion, an opening 13 being substantially shaped in conformity to the periphery of the eye, the opening 13 having an outward extending rim 131, a substantially funnel shaped cup body 12 extending between the opening 13 and the bottom 11, the cup body 12 having two series of raised protuberances 121, and a separate suction loop member 132 shaped to assemble with the rim 131 by snapping.

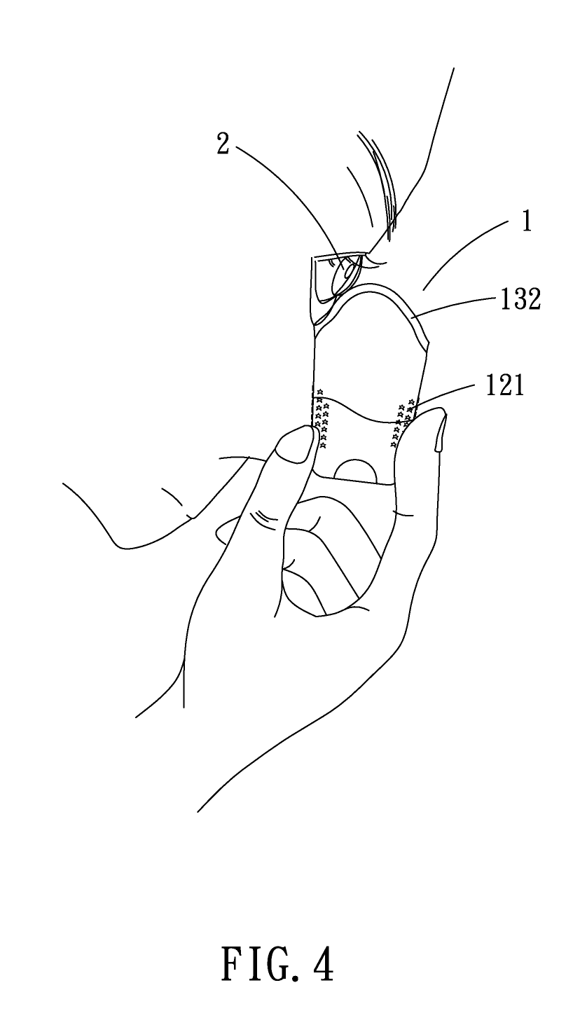

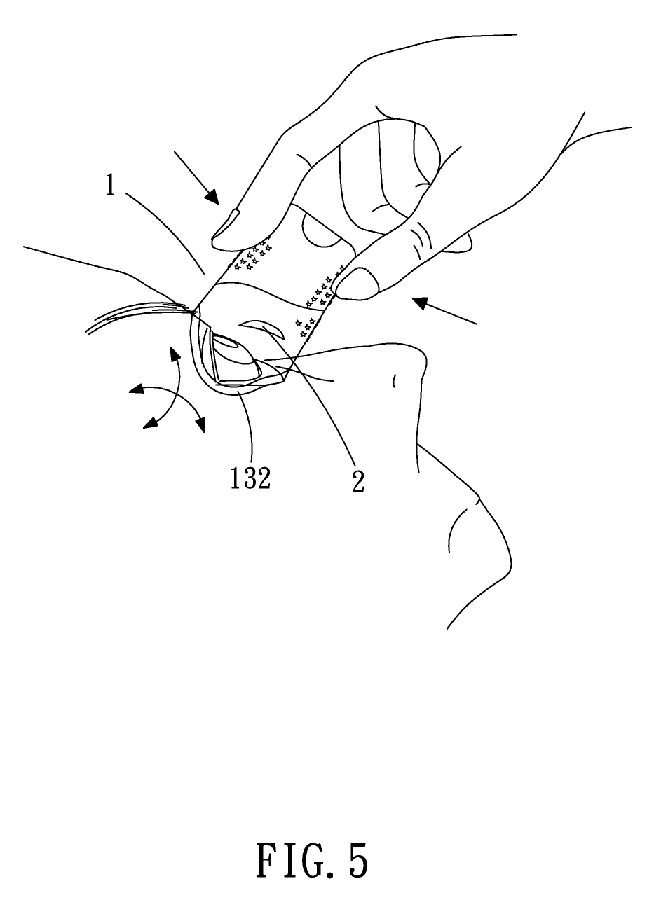

A lens removal operation of the invention will be described in detail below by referring to FIGS. 4 to 6 specifically. First, assemble the suction loop member 132 and the rim 131 together. The cup 1 may work similar to a conventional suction cup. A user may pour an amount of IV (intravenous) fluid into the cup 1. Next, the user may hold the cup 1 by grasping the series of raised protuberances 121 with the thumb and the index finger (i.e., with the purpose of increasing the traction between the cup body 12 and the skin of the fingers). Next, the user may engage the suction loop member 132 with the periphery of the eye. Thus, the suction loop member 132 adheres to the periphery of the eye. Next, the user may rotate his or her head to have the face upward and the cup 1 is thus upside down. Hence, a contact lens (e.g., soft lens) 2 placed on the cornea of the eye is immersed in the IV fluid. The IV fluid may decrease the friction between the lens 2 and the cornea of the eye. Also, the user may press the cup 1 and then release the cup 1 for a plurality of times in a short period of time. This has the effect of quickly changing the internal pressure of the cup 1. Moreover, the user may rub and knead the muscles around the eyes (see arrows in FIG. 5). Further, the user may rotate the eyeball and/or blink. As a combination of two or more of the above actions, the lens 2 disengages from the cornea of the eye. Next, the user may rotate his or her head to have the face downward and the lens 2 thus falls onto the half-spherical projection 111 for anchoring. Next, the user may pull the cup 1 to disengage from the eye. Finally, the user may pick up the lens 2 from the cup 1 for storage or cleaning.

Preferably, the half-spherical projection 111 and the lens 2 are complementary members each other.

It is envisaged by the invention that finger contact with the eye is avoided. Hence, it is sanitary.

While the invention herein disclosed has been described by means of specific embodiments, numerous modifications and variations could be made thereto by those skilled in the art without departing from the scope and spirit of the invention set forth in the claims.

What is claimed is:

1. A method of removing a contact lens by means of a device including a flexible cup comprising a projection on a bottom and an outward extending rim on an opening, and a suction loop member shaped to assemble with the rim by snapping, comprising the steps of:
pouring an amount of IV (intravenous) fluid into the cup;
adhering the suction loop member to a periphery of the eye by engaging;
immersing the contact lens placed on the cornea of the eye in the IV fluid by inverting the cup; and
repeatedly pressing and releasing the cup until the contact lens falls onto the projection.

2. The method of claim 1, wherein the projection is half-spherical.

3. The method of claim 1, wherein the projection is on a center of the bottom of the cup.

4. The method of claim 1, wherein the cup further comprises two opposite series of raised protuberances on a peripheral surface between the bottom thereof and the rim.
